(19) United States
(12) Patent Application Publication
(10) Pub. No.: US 2020/0188453 A1
(43) Pub. Date: Jun. 18, 2020

(54) METHOD OF A LACTOBACILLUS PLANTARUM TWK10 COMPOSITION FOR IMPROVING INFLAMMATION AFTER EXERCISE

(71) Applicant: SYNBIO TECH INC., Kaohsiung City (TW)

(72) Inventors: Chi-Chang Huang, Taoyuan City (TW); Wen-Ching Huang, Taipei City (TW); Jin-Sheng Lin, Kaohsiung City (TW); Mon-Chien Lee, Taoyuan City (TW); Ker-Sin Ng, Kaohsiung City (TW)

(21) Appl. No.: 16/217,408

(22) Filed: Dec. 12, 2018

(51) Int. Cl. A61K 35/747; A23L 33/135; C12N 1/20; C12R 1/225

(52) U.S. Cl. CPC A61K 35/747; A23L 33/135; A23V 2002/00; C12R 1/225; C12N 1/20

(57) ABSTRACT

The invention provides a method which comprises administering an effective amount of a probiotic composition including Lactobacillus plantarum TWK10 for improving inflammation after exercise. The Lactobacillus plantarum TWK10 is deposited in Taiwan Food Industry Research and Development Institute (FIRDI) with the accession number BCRC910734, and in China General Microbiological Culture Collection Center (CGMCC) with the accession number CGMCC 13008. The probiotic composition comprises pharmaceutically acceptable carrier, excipients and diluent, and a dosage form is selected from the group consisting of solution, suspension, emulsion, powder, tablet, pill, syrup, lozenge, troche, chewing gum, jatex and capsule, and can be further made into fluid milk products, concentrated milk, yogurt, sour milk, frozen yogurt, Lactobacillus-fermented beverages, milk powder, ice cream, cream cheeses, hard cheeses, soy milk, fermented soy milk, vegetable-fruit juices, fruit juices, sports drinks, confectionery, jellies, candies, infant foods, health foods, animal feeds, Chinese medicinal herbs compositions and dietary supplements.

### FIELD OF THE INVENTION

The present invention belongs to the application field of probiotics, and more particularly relates to Lactobacillus plantarum that can be used for anti-inflammation after exercise.

### DESCRIPTION OF THE PRIOR ART

Probiotics are very popular in the market of healthy foods, because the source of probiotics is natural and harmless to the human body, the threshold for taking them is low, and they provide various regulating effects on human body's health. Among them, Lactobacillus plantarum is a common probiotic, a Gram-positive bacterium, which can be found in fermented foods or animal saliva. It has the largest genome in Lactobacilli and has many variations, so it often demonstrates different characteristics and effects among the same type of strain due to the extent of variation and the condition of adaptation.

Previous reports have indicated that Lactobacillus plantarum has a variety of medical uses, including protection of respiratory tract against viral infections, increasing the sensitivity of cancer cells to chemotherapeutic drugs, as well as an additive for various foods, for example, for preventing the growth of toxic fungi in corns.

Inflammation refers to the defensive-based response of live tissues with vascular system to inflammatory factors and local damage, which can be divided into acute inflammation and chronic inflammation. Acute inflammation is the initial response of organisms to harmful stimuli. More plasma and leukocytes (especially granulocytes) move from the blood to the damaged tissues for a series of chain reactions involving local vascular system, immune system and individual cell in the damaged tissues. Chronic inflammation leads to changes in cell types in the inflamed area, and causes synchronous tissue destruction and repair. In addition, human adipocyte has the ability to secrete inflammatory hormones, so excessively high body adipocyte is also a factor that causes chronic inflammation of the body. In some special cases, inflammation may even cause allergies in the human body's immune system, which in turn attacks its own tissues and cells, such as rheumatoid arthritis, lupus erythematosus and other immune system allergies. Long-term chronic inflammation can cause a series of diseases and make the organs and tissues gradually lose function, not only cause fatigue, aging, hormonal imbalance easily, but also lead to a variety of serious diseases, including hepatitis, pneumonia, nephritis, autoimmune diseases, arthritis, diabetes, Alzheimer's disease, cardiovascular disease, and even cancers.

Injury to muscle fibers or connective tissues from exercise can cause muscle inflammation, accompanied by ache, pain, various discomforts, and chronic inflammation caused by long-term accumulation, which not only discourage people from exercising and reduce quality of life, it also causes the human body to be prone to further sports injuries and posture changes. Especially for athletes, how to effectively reduce inflammatory conditions after exercise is a key factor to improve the efficiency of practice and competition and reduce sports injuries.

Although there are a lot of health foods on the market in Taiwan, only five have been certified by Taiwan Ministry of Health and Welfare to have anti-inflammatory effects (Table 1), with the same purpose of adjusting allergic physique and antianaphylaxis. However, the mechanism of allergies is induced by allergens, resulting in immunoglobulin to over-stimulate the sensitization of mast cells; unlike acute or chronic inflammation after exercise, which is mainly caused by injury on the tendon or ligament. At present, the application of probiotics to improve inflammation after exercise remains to be studied and researched.

TABLE 1
List of anti-inflammatory health foods approved by Taiwan Food
and Drug Administration of the Ministry of Health and Welfare.

| License Number | Product Name | Health Caring Effects | Applicant |
|---|---|---|---|
| Health Food License No. | Stolle Essential (Powder | Auxiliary adjustment of | LanFar International Co., |
| A00116 by Taiwan | Food) | allergic physique, | Ltd. |
| Department of Health | | immunomodulatory | |
| | | function | |
| Health Food License No. | Yakult 300LIGHT Live | Auxiliary adjustment of | Yakult Co., Ltd. |
| A00128 by Taiwan | Bacterial Fermented Milk | allergic physique, | |
| Department of Health | | immunomodulatory | |
| | | function, improvement of | |
| | | gastrointestinal function | |
| Health Food License No. | Baiyimin Probiotic | Auxiliary adjustment of | Syngen Biotech Co., Ltd. |
| A00218 by Taiwan | Capsule | allergic physique | |
| Department of Health | | | |
| Health Food License No. | L-137tment oLactobacilli | Auxiliary adjustment of | Chambio Co., Ltd. |
| A00322 by Taiwan | Capsule | allergic physique, | |
| Department of Health | | immunomodulatory | |
| | | function | |
| Health Food License No. | Roymin Capsule | Auxiliary adjustment of | Standard Chem & Pharm |
| A00329 by Taiwan | | allergic physique | Co., Ltd. |
| Department of Health | | | |

Previously, related literatures disclose that Lactobacillus plantarum can be used to improve anti-inflammatory related diseases; however, there is no related publication of probiotics that can improve inflammation after exercise.

Therefore, how to provide a new method of probiotics to improve inflammation after exercise is the problem to be solved in the present invention.

### SUMMARY OF THE INVENTION

The present invention provides a method for improving inflammation after exercise which comprises administering an effective amount of a probiotic composition including Lactobacillus plantarum TWK10. The Lactobacillus plantarum TWK10 is deposited in Taiwan Food Industry Research and Development Institute (FIRDI) with the accession number BCRC910734, and is also deposited in China General Microbiological Culture Collection Center (CGMCC) with the accession number CGMCC 13008.

In order to achieve the aforementioned objective of the present invention, improving inflammation after exercise by the Lactobacillus plantarum TWK10 is achieved by reducing a neutrophil to lymphocyte ratio (NLR) and a platelet to lymphocyte ratio (PLR).

In order to achieve the aforementioned objective of the present invention, the Lactobacillus plantarum TWK10 is a live or an inactivated bacterium, and the inactivated bacterium is inactivated by heat or subjected to other physical or chemical treatments. When the Lactobacillus plantarum TWK10 is a live bacterium, a dose is greater than or equal to 3×10¹⁰ CFU (colony forming unit)/day; wherein a dose greater than 1×10¹¹ CFU (colony forming unit) per day has better effects than 3×10¹⁰ CFU on improving inflammation after exercise.

The present invention further provides a method for improving diseases caused by excessively high neutrophil to lymphocyte ratio (NLR) and platelet to lymphocyte ratio (PLR) by administering an effective amount of a probiotic composition, and the probiotic composition comprises the Lactobacillus plantarum TWK10 with the accession number BCRC910734 (CGMCC 13008).

The aforementioned probiotic composition can comprise pharmaceutically acceptable carrier, excipients and diluent, and a dosage form of the composition is selected from the group consisting of solution, suspension, emulsion, powder, tablet, pill, syrup, lozenge, troche, chewing gum, jatex and capsule.

The aforementioned probiotic composition can be further made into fluid milk products, concentrated milk, yogurt, sour milk, frozen yogurt, Lactobacillus-fermented beverages, milk powder, ice cream, cream cheeses, hard cheeses, soy milk, fermented soy milk, vegetable-fruit juices, fruit juices, sports drinks, confectionery, jellies, candies, infant foods, health foods, animal feeds, Chinese medicinal herbs compositions and dietary supplements.

The aforementioned probiotic composition comprises one or any combination of the following probiotic groups: Lactobacillus species, Lactococcus species, Pediococcus species, Leuconostoc species, Enterococcus species, Streptococcus species, Bifidobacterium species, and Saccharomycete species.

An embodiment of the present invention demonstrates that the live and high-dose group (3×10¹¹ CFU/day) and the heat-killed group of TWK10 can reduce the inflammatory index values (NLR and PLR) and help to improve inflammation after exercise, and is available to be applied to diseases or symptoms caused by excessively high NLR and PLR.

The advantages of the new method of the probiotics provided in the present invention are as follows:

1) The Objects are Consice and Novel

For the first time, TWK10 is provided for improving the symptoms of inflammation after exercise, which is different from other probiotics that inhibit diseases caused by inflammation, and providing evidence of rigorous human clinical data; especially for athletes, it can help enhance physical functions in all aspects.

2) Wide Range of Applications

Because inflammation is the cause or evaluation factor of common diseases or symptoms, the method of the probiotics in the present invention will help to improve the diseases and symptoms caused by inflammation.

### BRIEF DESCRIPTION OF THE DRAWINGS

FIG. 1 shows the change in neutrophil to lymphocyte ratio (NLR) after exercise and administration of a probiotic composition containing Lactobacillus plantarum TWK10;

FIG. 2 shows the change in platelet to lymphocyte ratio (PLR) ratio after exercise and administration of the probiotic composition containing Lactobacillus plantarum TWK10.

### DETAILED DESCRIPTION OF THE INVENTION

The present invention will be further described in the following embodiments. However, the embodiments are merely representative and illustrative of the meaning and spirit of the present invention, and thus the embodiments are for exemplified instead of limitative purpose, and should not be construed as the limitations of implementing the present invention or being limited to the described embodiments or manners.

All technical and scientific terms described in the specification and claims are defined according to the following description unless otherwise defined. Among them, the singular terms “a”, “an”, “one”, “the” can refer to more than one subject unless otherwise indicated; unless otherwise indicated, ordinary techniques employed in the implementation of the present invention can include clinical sample collection and analysis, high-efficient liquid-phase chromatography, protein chemistry, biochemistry, recombinant DNA technology, pharmacological technology; in addition, the terms “contain”, “include” and “comprise” are open conjunctions; unless otherwise indicated, the materials used in the present invention are commercially available and can be easily acquired.

The term “probiotics” refers to microorganisms that provide physiological health benefits to a user when properly administered. The microorganisms are non-toxic and have intestinal adhesion, and gain effects; the term “colony forming unit (CFU)” refers to a term of total number of colonies of microorganisms such as bacteria, mold, yeast, etc. per unit volume; the term “composition” refers to a product which is derived from mixing or combining more than one active ingredient; “carrier”, “excipients” refer to a compound or medicament that is non-toxic, and has the function of assisting cells or tissues to absorb drugs, and can be aromatics, buffers, binders, colorants, disintegrants, thinners, emulsifiers, extenders, flavor-improving agents, gellants, glidants, preservatives, skin-penetration enhancers, solubilizers, stabilizers, suspending agents, sweeteners, tonicity agents, viscosity-increasing agents, or any combination of the above; the term “diluent” refers to a solvent used for diluting prior to administration of the composition, and is used for stabilizing the compound. Buffer solution in the present invention is used to dissolve salt and regulate pH value, including but not limited to phosphate buffered saline (PBS); the term “effective amount” refers to a sufficient amount of a compound or drug to reduce one or more disease symptoms or physiological conditions of a patient after taking it. For example, an “effective amount” of a treatment includes a dose of a compound provided in the present invention that clinically reduces a particular physiological indicator significantly; the term “maximal oxygen uptake (abbreviated as VO₂max)” refers to a highest value of oxygen that can be consumed or utilized by tissue cells when a person is engaged in the most intense exercise above sea level; the term “endurance exercise ability test with fixed intensity and time” refers to exercise testing on a treadmill or bicycle with an intensity of 60% VO₂max or more for at least 30 minutes.

The following embodiments are merely illustrative of the probiotic composition used in the present invention, wherein the probiotics can be live or inactivated, and the inactivated bacteria are inactivated by heat or subjected to other physical or chemical treatments.

The Lactobacillus plantarum strain TWK10 (hereinafter referred to as TWK10) used in the present invention was obtained by separating from fermented cabbages in Taiwan by Dr. Tsung-Yu Tsai, and is deposited in China General Microbiological Culture Collection Center (CGMCC) with the accession number CGMCC 13008, and the juridical association of Taiwan Food Industry Research and Development Institute (FIRDI) with the accession number BCRC910734.

Anti-inflammatory effects of Lactobacillus plantarum TWK10

a) Experimental Design and Process

The experiment employs a double-blind cross-over trial. The testees were between 20-40 years old. After a two-week wash period, they were administrated by the probiotics for six weeks. The testees were divided into 5 groups, 16 people in each group, according to the result of a maximum oxygen uptake (VO₂max, mL/kg/min), and an endurance exercise test with fixed intensity at the beginning and end of the administration is executed. Furthermore, a blood test to determine changes of physiological factors was also performed. The probiotic composition of each group was taken three times a day, one capsule at a time, and a dose of administration and strain of each group are listed as follows:

(1) Placebo group: TWK10-free placebo capsules (containing maltodextrin, lactose, microcrystalline α-cellulose), 3 capsules a day, take one capsule after each of three meals with warm water.

(2) Low-dose group: recommended 1 time dose of TWK10 live bacteria group (TWK10-L): One TWK10 capsule containing 1×10¹⁰ CFU, 3 capsules per day, 3×10¹⁰ CFU/day, take one capsule after each of three meals with warm water.

(3) Middle-dose group: recommended 3 times dose of TWK10 live bacteria group (TWK10-M): One TWK10 capsule containing 3×10¹⁰ CFU, 3 capsules per day, 9×10¹⁰ CFU/day, take one capsule after each of three meals with warm water.

(4) High-dose group: recommended 10 times dose of TWK10 live bacteria group (TWK10-H): One TWK10 capsule containing 1×10¹¹ CFU, 3 capsules per day, 3×10¹¹ CFU/day, take one capsule after each of three meals with warm water.

(5) TWK10 heat-killed bacteria group (TWK10-HK): One TWK10 heat-killed bacteria capsule containing 1×10¹¹ CFU, 3 capsules per day, 3×10¹¹ CFU/day, take one capsule after each of three meals with warm water.

b) Results

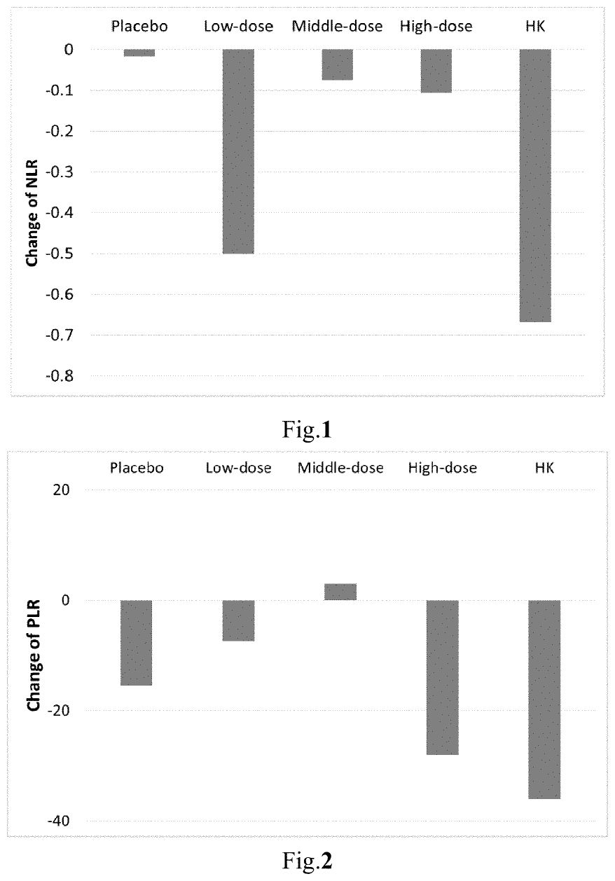

After analysis of six weeks that the testees had performed the endurance exercise test with fixed intensity and time (120 minutes), the change was represented in neutrophil to lymphocyte ratio (NLR) and platelet to lymphocyte ratio (PLR) in the respective groups, both of which representing the state of the lymphatic system in the body and being indicators for measuring inflammation in the body. A decrease in a ratio indicates an improvement in inflammation, and there are even reports indicating that NLR or PLR can be used as an observation factor for cancer metastasis or other diseases. As shown in the results in FIG. 1 and FIG. 2, the vertical axis indicates the amount of the change in NLR or PLR relative to the start of the test.

The results show that High-dose, Medium-dose, Low-dose groups and TWK10 heat-killed bacteria group can reduce the NLR. Among the change of NLR, the low-dose and heat-killed bacteria groups decreased by about 30-40 times compared with the control group. As for PLR, the High-dose and TWK10 heat-killed bacteria groups decreased by 1.2-1.8 times compared with the control group. Overall, taking TWK10 dead bacteria can improve inflammation after exercise, and daily dose of TWK10 live bacteria of more than 3×10¹⁰ CFU also has effects of improving inflammation after exercise, if taken higher dose per day, which is more than 9×10¹⁰ CFU live bacteria, for example, 1×10¹¹ or 3×10¹¹ CFU, better effects can be achieved.

In summary, TWK10 has the effects of reducing inflammation after exercise. In addition, since TWK10 can reduce NLR or (and) PLR, NLR and PLR can also be used as indicators and causes for the diagnosis of metastasis and infections of many cancers and diseases. The implementation of any use of the results of the present invention to improve related diseases or symptoms caused by excessively high NLR, or PLR, shall be deemed to be included in the scope of the claims of the present invention.

A dosage form of the probiotic composition used in the present invention comprises, but is not limited to, solution, emulsion, suspension, powder, tablet, pill, lozenge, troche, chewing gum, capsule, and other similar or suitable dosage forms.

The probiotic composition used in the present invention can optionally comprises other bacteria species taught in the general knowledge in the technical field of the present invention: Lactobacillus sp., Lactococcus sp., Pediococcus sp., Leuconostoc sp., Enterococcus sp., Streptococcus sp., Bifidobacterium sp., yeasts (Saccharomyces sp.).

The probiotic composition used in the present invention can be a food composition, and further made into capsule, tablet, beverages, powder or dairy products; the aforementioned composition used in the present invention can further be added with an edible material to prepare as a food product or health caring product, wherein the edible material comprises, but is not limited to: water, fluid milk products, milk, concentrated milk, fermented milk (such as yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, jellies, candies, infant formulas, health foods, animal feeds, Chinese medicinal herbs, and dietary supplements.

The aforementioned composition used in the present invention can be a dietary supplement which can be administered to a user in the following manners: mixed with a suitable drinkable liquid, such as water, yoghurt, milk or fruit juices; or can be mixed with a solid or liquid food. The form of the dietary supplement can be in an administrated form of tablet, pill, capsule, lozenge, granule, powder, suspending agent, sachet, soft tablet, candy, bar, syrup and corresponding administration forms, generally in the form of dose unit and is manufactured by conventional methods for preparing dietary supplements.

In the embodiments of the present invention, an effective dose varies depending on the combination therapy. The combination therapy can further include periodic treatments that vary depending on factors such as co-administered compositions, diseases, physiological abnormalities, physiological conditions, and the like.

The embodiments described above are merely for explaining the technical ideas and characteristics of the present invention, and the purpose thereof is to enable those skilled in the art to understand the contents of the present invention and implement them accordingly; they are not intended to limit the scope of the claims of the present invention, that is, any equivalent changes or modifications made according to the spirit of the disclosure should still be covered by the claims of the present invention.

What is claimed is:

1. A method for improving inflammation after exercise which comprises administering an effective amount of a probiotic composition including Lactobacillus plantarum TWK10, wherein the Lactobacillus plantarum TWK10 is deposited in China General Microbiological Culture Collection Center (CGMCC) with an accession number CGMCC 13008.

2. The method as claimed in claim 1, wherein improving inflammation after exercise is achieved by reducing a neutrophil to lymphocyte ratio (NLR) or a platelet to lymphocyte ratio (PLR).

3. The method as claimed in claim 1, wherein the Lactobacillus plantarum TWK10 is live or inactivated bacteria inactivated by heat or subjected to other physical or chemical treatments.

4. The method as claimed in claim 3, wherein a dose is greater than or equal to 3×10¹⁰ CFU (colony forming unit)/day while the Lactobacillus plantarum TWK10 is live.

5. The method as claimed in claim 4, wherein a dose greater than 1×10¹¹ CFU (colony forming unit) per day has better effects than a dose of 3×10¹⁰ CFU on improving inflammation after exercise.

6. The probiotic composition as claimed in claim 1, the probiotic composition comprising pharmaceutically acceptable carrier, excipients and diluent.

7. The probiotic composition as claimed in claim 1, wherein a dosage form of the probiotic composition is selected from the group consisting of solution, suspension, emulsion, powder, tablet, pill, syrup, lozenge, troche, chewing gum, jatex and capsule.

8. The probiotic composition as claimed in claim 1, wherein the probiotic composition is made into fluid milk products, concentrated milk, yogurt, sour milk, frozen yogurt, Lactobacillus-fermented beverages, milk powder, ice cream, cream cheeses, hard cheeses, soy milk, fermented soy milk, vegetable-fruit juices, fruit juices, sports drinks, confectionery, jellies, candies, infant foods, health foods, animal feeds, Chinese medicinal herbs compositions and dietary supplements.

9. The probiotic composition as claimed in claim 1, wherein the probiotic composition comprises one or any combination of the following probiotic groups: Lactobacillus species, Lactococcus species, Pediococcus species, Leuconostoc species, Enterococcus species, Streptococcus species, Bifidobacterium species, and Saccharomycete species.

10. A method for improving diseases or symptoms caused by excessively high neutrophil to lymphocyte ratio (NLR) and platelet to lymphocyte ratio (PLR) by administering an effective amount of a probiotic composition, wherein the probiotic composition comprising Lactobacillus plantarum TWK10 with an accession number CGMCC 13008.
